(19) United States
(12) Patent Application Publication
(10) Pub. No.: US 2010/0286285 A1
(43) Pub. Date: Nov. 11, 2010

(54) Pharmaceutical composition comprising oleaginous ointments and vitamin D or its derivatives in the solubilized state

(75) Inventors: Nathalie Barthez, Nice (FR); Sandrine Orsoni, Mandelieu (FR); Laurent Fredon, Roquefort Les Pins (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, BIOT (FR)

(21) Appl. No.: 11/984,392

(22) Filed: Nov. 16, 2007

Related U.S. Application Data
(63) Continuation of application No. PCT/FR2006/000971, filed on Apr. 28, 2006.

(30) Foreign Application Priority Data
May 16, 2005 (FR) 05/04889
Nov. 23, 2006 (WO) 2006/123031 A2

(51) Int. Cl. A61K 31/085; A61P 17/06

(52) U.S. Cl. 514/721

(57) ABSTRACT

The present invention relates to an anhydrous pharmaceutical composition for use in the treatment of psoriasis and other skin disorders, characterized in that it comprises an oleaginous ointment and, as active ingredient, a compound chosen from vitamin D and its derivatives, said active agent being in a solubilized form in said composition.

The present invention relates to the field of the formulation of active ingredients for the purpose of topical pharmaceutical application.

The present invention relates more particularly to a stable, anhydrous pharmaceutical composition comprising an oleaginous ointment and, as active ingredient, a compound chosen from vitamin D and its derivatives, and to its use for the topical treatment of psoriasis and other skin disorders.

Vitamin D and its derivatives are generally used in dermatology in the treatment of psoriasis since they limit the excessive production of skin cells on the surfaces affected and possess proven advantages for the treatment of this condition, which is characterized in particular by the presence of thick, squamous, dry lesions.

Since vitamin D and its derivatives are highly unstable in aqueous media, it is advisable to formulate these active ingredients in compositions of anhydrous type. The anhydrous compositions currently available, which allow the formulation of water-sensitive active ingredients, are generally ointment-type compositions consisting mainly of petroleum jelly.

Now, such compositions either contain a high percentage of petroleum jelly in order to prevent the occlusiveness and the penetration of the active agent, or contain a high percentage of propenetrating glycol—at least 20%—in order to promote the penetration of the active agent, but are tacky and can cause problems of intolerance (see the article “The critical role of the vehicle to therapeutic efficacy and patient compliance”, Piacquadio et al., J. Am. Acad. Dermatol., August 1998).

One of the aims of the present invention is to provide an anhydrous pharmaceutical composition of ointment type, which has good stability and good tolerance, and which allows optimized release of the active agent, while at the same time being less tacky and less greasy on application.

A subject of the present invention is therefore an anhydrous pharmaceutical composition, characterized in that it comprises:

a) an oleaginous ointment comprising petroleum jelly and a combination of emollients comprising at least one liquid fatty substance and at least one butter, and

b) as active ingredient, a compound chosen form vitamin D and its derivatives, of general formula (I) below:

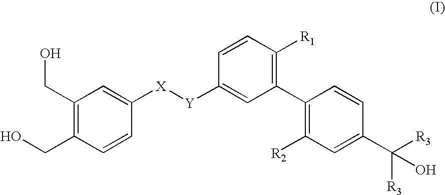

in which:
X—Y represents a bond chosen from the following structures:

—CH₂—CH₂—

—CH₂—O—

—O—CH₂—

—CH₂—N(R₄)—

R₄ having the meanings given hereinafter,
R₁ represents a methyl radical or an ethyl radical,
R₂ represents an ethyl radical, a propyl radical or an isopropyl radical,
R₃ represents an ethyl radical or a trifluoromethyl radical,
R₄ represents a hydrogen atom, a methyl radical, an ethyl radical or a propyl radical,

said active agent being in a solubilized form in said composition.

Such a composition is for topical application and makes it possible to overcome the abovementioned drawbacks.

The term “solubilized form” is intended to mean a dispersion in the molecular state in a liquid, no crystallization of the active agent being visible to the naked eye or even under a cross-polarization optical microscope.

For the purposes of the present invention, the term “anhydrous composition” is intended to mean a composition substantially free of added water, i.e. having a water content of less than or equal to 5% by weight relative to the total weight of the composition, in particular less than or equal to 3%, preferably equal to zero.

Such a composition is in particular for use in the treatment of psoriasis and other skin disorders. The expression “skin disorders other than psoriasis” is intended to mean in particular atopic dermatitis, contact dermatitis and seborrhoeic dermatitis. Preferably, the composition according to the present invention is for use in the treatment of psoriasis.

Such a composition is in particular intended for topical application.

The active ingredients that can be used in the compositions according to the invention are vitamin D and its derivatives of formula (I), used alone or as a mixture.

The term “vitamin D” is intended to mean the various forms of vitamin D, such as, for example, vitamin D₂ or vitamin D₃.

The vitamin D derivatives used according to the invention are described in the application WO 03/050067. They are compounds that are structural analogues of vitamin D and that show a selective activity on cell proliferation and differentiation.

Among the compounds of formula (I) which fall within the context of the present invention, mention may in particular be made of the following:
1—{5-[4′-(1-ethyl-1-hydroxypropyl)-6-methyl-2′-propylbiphenyl-3-yloxymethyl]-2-hydroxy-methylphenyl}methanol;
2—{5-[6,2′-diethyl-4′-(1-ethyl-1-hydroxy-propyl)biphenyl-3-yloxymethyl]-2-hydroxy-methylphenyl}methanol;
3—{4-[6-ethyl-4′-(1-ethyl-1-hydroxypropyl)-2′-propylbiphenyl-3-yloxymethyl]-2-hydroxy-methylphenyl}methanol;
4—{4-[6-ethyl-4′-(1-ethyl-1-hydroxypropyl)-2′-isopropylbiphenyl-3-yloxymethyl]-2-hydroxy-methylphenyl}methanol;
5—(4-{2-[4′-(1-ethyl-1-hydroxypropyl)-6-methyl-2′-propylbiphenyl-3-yl]ethyl}-2-hydroxy-methylphenyl)methanol;
6—{4-[4′-(1-ethyl-1-hydroxypropyl)-6-methyl-2′-propylbiphenyl-3-ylmethoxy]-2-hydroxymethyl-phenyl}methanol;
7—(4-{[4′-(1-ethyl-1-hydroxypropyl)-6-methyl-2′-propylbiphenyl-3-ylamino]methyl}-2-hydroxymethylphenyl)methanol;
8—[4-({[4′-(1-ethyl-1-hydroxypropyl)-6-methyl-2′-propylbiphenyl-3-yl]methylamino}methyl)-2-hydroxymethylphenyl]methanol;
9—[4-({ethyl-[4′-(1-ethyl-1-hydroxypropyl)-6-methyl-2′-propylbiphenyl-3-yl]amino}methyl)-2-hydroxymethylphenyl]methanol;
10—[4-({[4′-(1-ethyl-1-hydroxypropyl)-6-methyl-2′-propylbiphenyl-3-yl]propylamino}methyl)-2-hydroxymethylphenyl]methanol;
11—(2-hydroxymethyl-4-{2-[6-methyl-2′-propyl-4′-(2,2,2-trifluoro-1-hydroxy-1-trifluoromethyl-ethyl)biphenyl-3-yl]ethyl}phenyl)methanol;
12—{2-hydroxymethyl-4-[6-methyl-2′-propyl-4′-(2,2,2-trifluoro-1-hydroxy-1-trifluoromethyl-ethyl)biphenyl-3-yloxymethyl]phenyl}methanol;
13—{2-hydroxymethyl-4-[6-methyl-2′-propyl-4′-(2,2,2-trifluoro-1-hydroxy-1-trifluoromethyl-ethyl)biphenyl-3-ylmethoxy]phenyl}methanol;
14—(2-hydroxymethyl-4-{[6-methyl-2′-propyl-4′-(2,2,2-trifluoro-1-hydroxy-1-trifluoromethyl-ethyl)biphenyl-3-ylamino]methyl}phenyl)-methanol;
15—[2-hydroxymethyl-4-({N-methyl[6-methyl-2′-propyl-4′-(2,2,2-trifluoro-1-hydroxy-1-trifluoromethylethyl)biphenyl-3-yl]amino}-methyl)phenyl]methanol;
16—[4-({N-ethyl[6-methyl-2′-propyl-4′-(2,2,2-trifluoro-1-hydroxy-1-trifluoromethylethyl)-biphenyl-3-yl]amino}methyl)-2-hydroxymethyl-phenyl]methanol;
17—[2-hydroxymethyl-4-({[6-methyl-2′-propyl-4′-(2,2,2-trifluoro-1-hydroxy-1-trifluoromethyl-ethyl)biphenyl-3-yl]N-propylamino}methyl)-phenyl]methanol;
18—(4-{2-[6-ethyl-4′-(1-ethyl-1-hydroxypropyl)-2′-propylbiphenyl-3-yl]ethyl}-2-hydroxy-methylphenyl)methanol;
19—{4-[6-ethyl-4′-(1-ethyl-1-hydroxypropyl)-2′-propylbiphenyl-3-ylmethoxy]-2-hydroxymethyl-phenyl}methanol;
20—(4-{[6-ethyl-4′-(1-ethyl-1-hydroxypropyl)-2′-propylbiphenyl-3-ylamino]methyl}-2-hydroxy-methylphenyl)methanol;
21—[4-({[6-ethyl-4′-(1-ethyl-1-hydroxypropyl)-2′-propylbiphenyl-3-yl]methylamino}methyl)-2-hydroxymethylphenyl]methanol;
22—[4-({ethyl-[6-ethyl-4′-(1-ethyl-1-hydroxy-propyl)-2′-propylbiphenyl-3-yl]amino}methyl)-2-hydroxymethylphenyl]methanol;
23—[4-({[6-ethyl-4′-(1-ethyl-1-hydroxypropyl)-2′-propylbiphenyl-3-yl]propylamino}methyl)-2-hydroxymethylphenyl]methanol;
24—(4-{2-[6-ethyl-2′-propyl-4′-(2,2,2-trifluoro-1-hydroxy-1-trifluoromethylethyl)biphenyl-3-yl]ethyl}-2-hydroxymethylphenyl)methanol;
25—{4-[6-ethyl-2′-propyl-4′-(2,2,2-trifluoro-1-hydroxy-1-trifluoromethylethyl)biphenyl-3-yloxymethyl]-2-hydroxymethylphenyl}methanol;
26—{4-[6-ethyl-2′-propyl-4′-(2,2,2-trifluoro-1-hydroxy-1-trifluoromethylethyl)biphenyl-3-ylmethoxy]-2-hydroxymethylphenyl}methanol;
27—(4-{[6-ethyl-2′-propyl-4′-(2,2,2-trifluoro-1-hydroxy-1-trifluoromethylethyl)biphenyl-3-ylamino]methyl}-2-hydroxymethylphenyl)-methanol;
28—{4-({[6-ethyl-2′-propyl-4′-(2,2,2-trifluoro-1-hydroxy-1-trifluoromethylethyl)biphenyl-3-yl]methylamino}methyl)-2-hydroxymethyl-phenyl]methanol;
29—[4-({N-ethyl[6-ethyl-2′-propyl-4′-(2,2,2-trifluoro-1-hydroxy-1-trifluoromethylethyl)-biphenyl-3-yl]amino}methyl)-2-hydroxymethyl-phenyl]methanol;
30—[4-({[6-ethyl-2′-propyl-4′-(2,2,2-trifluoro-1-hydroxy-1-trifluoromethylethyl)biphenyl-3-yl]-N-propylamino}methyl)-2-hydroxymethyl-phenyl]methanol;
31—(4-{[4′-(1-ethyl-1-hydroxypropyl)-6,2′-dimethylbiphenyl-3-ylamino]methyl}-2-hydroxymethylphenyl)methanol.

Particularly preferably, the vitamin D derivative used in the present invention is {4-[6-ethyl-4′-(1-ethyl-1-hydroxypropyl)-2′-propylbiphenyl-3yloxymethyl]-2-hydroxymethylphenyl}methanol (compound 3—above) of formula (II) below:

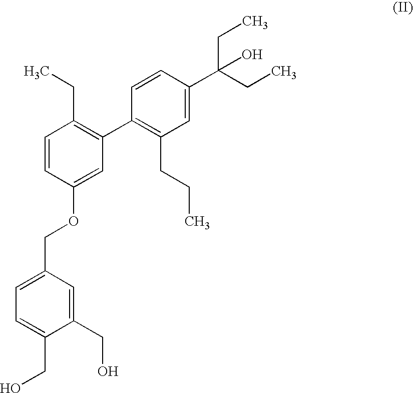

The compositions of the invention are found to be particularly effective for preserving a satisfactory chemical stability of the active ingredient sensitive to oxidation, to water and to aqueous media in general.

The term “satisfactory chemical stability” is intended to mean a composition which, over a period of at least three months, respectively at ambient temperature and at 40° C.:
does not show any substantial modification of its macroscopic appearance,
comprises an active ingredient content of at least 90%, and more particularly of at least 95%, of the initial content by weight.

Advantageously, the amount of active ingredient, i.e. of vitamin D and/or its derivatives and in particular of {4-[6-ethyl-4′-(1-ethyl-1-hydroxypropyl)-2′-propyl-biphenyl-3-yloxymethyl]-2-hydroxymethylphenyl}methanol, in a solubilized form in the composition according to the invention is from 0.0001 to 5% by weight relative to the total weight of the composition, preferably from 0.001% to 1% by weight, and more particularly from 0.05% to 0.2% by weight.

More particularly, the vitamin D and/or its derivatives, in particular {4-[6-ethyl-4′-(1-ethyl-1-hydroxypropyl)-2′-propylbiphenyl-3-yloxymethyl]-2-hydroxymethylphenyl}methanol, which is part of the composition of the invention, is in the solubilized state in order to confer on the compositions of the invention good properties of release/penetration into the skin, this being allied with more advantageous kinetics. The expression “good release/penetration capacity” is intended to mean a good distribution of the composition of the invention, and therefore of the active ingredient that it contains, through the stratum corneum of the skin and also through the subcutaneous layers such as the epidermis and the dermis.

For the purpose of the present invention and according to the US Pharmacopoeia (“USP”), the term “ointment” is intended to mean a semi-solid preparation for external application to the skin or the mucous membranes. Ointments or unguents are used topically for many applications, for example as barrier creams, antiseptics, emollients, etc. Ointments are used for their emollient effect, they are simple to apply and readily penetrate the skin.

Five types of ointments commonly exist, differentiated on the basis of their physical composition. The most common type of ointment, which is that to which the present invention relates, is the oleaginous base ointment, referred to as “oleaginous ointment”; this ointment is anhydrous, hydrophobic, occlusive and comprises predominantly petroleum jelly.

According to an advantageous embodiment of the invention, the oleaginous ointment does not contain any aqueous phase and comprises in particular petroleum jelly and a combination of emollients comprising at least one liquid fatty substance and at least one butter. This combination confers very good tolerance on the formula, and allows optimized release of the active agent, while at the same time restoring the skin barrier impaired by the pathology. Moreover, a composition resulting from such a combination possesses good stability, while at the same time being less greasy and less tacky on application.

Petroleum jelly is a mixture of long-chain aliphatic hydrocarbons and is an excellent moisturizer. In fact, its occlusive properties allow the imperceptible transcutaneous loss of water to be blocked and the water to be trapped under the surface of the skin, by virtue of the formation of an inert occlusion membrane (“Effects of petrolatum on stratum corneum structure and function” Ghadially & all; Journal of the American Academy of Dermatology 1992; 26: 387-96). Petroleum jelly accelerates the recovery of the normal skin barrier properties in the case of skin affected by lesions, such as, for example, in atopic dermatitis or psoriasis. Furthermore, petroleum jelly is inert and therefore has no incompatibility at all, irrespective of the active ingredient.

In addition to petroleum jelly, the ointment comprises a first emollient consisting of at least one liquid fatty substance, the action of which is to make the skin supple and smooth and to promote the well-being of the skin. Such a product acts either by moisturizing the stratum corneum or by compensating for the insufficiency of the sebaceous secretion.

The term “liquid fatty substance” is intended to mean a lipophilic compound which is liquid at ambient temperature (25° C.) and ambient atmospheric pressure (760 mmHg).

As liquid fatty substances that stimulate the moisturization of the stratum corneum, mention may be made of oils, fatty alcohols, silicone oils, which slow down dehydration by virtue of an occlusive effect, but also humectants such as polyols, glycerol or urea. As liquid fatty substances that compensate for the insufficiency of sebaceous secretion, mention may be made of lipid products such as oils.

Oils are the preferred liquid fatty substances that can be used according to the invention; they are mineral, plant, animal or synthetic in nature.

As examples of mineral oils, mention may be made of liquid paraffins of various viscosities, such as Primol 352, Marcol 82 and Marcol 152 which are sold by the company Esso.

As plant oils, mention may be made of sweet almond oil, palm oil, soya oil, sesame oil and sunflower oil.

As animal oils, mention may be made of lanolin, squalene, fish oil and of mink oil.

As synthetic oils, mention may be made of an ester such as cetearyl isononanoate, for instance the product sold under the name Cetiol SN by the company Cognis France, diisopropyl adipate, for instance the product sold under the name Ceraphyl 230 by the company ISF, isopropyl palmitate, for instance the product sold under the name Crodamol IPP by the company Croda, and caprylic/capric triglyceride, such as Miglyol 812 sold by the company Huls/Lambert Riviere.

Advantageously, the liquid fatty substance that can be used in the present combination is chosen from liquid paraffin and sweet almond oil.

The amount of liquid fatty substance in the composition according to the invention is from 0.01% to 30% by weight relative to the total weight of the composition, preferably from 0.01% to 15% by weight. Preferably, the composition contains between 0.01% and 10% by weight of plant oil, and between 0.01% and 5% by weight of mineral oil.

Finally, in addition to the petroleum jelly and at least one liquid fatty substance, the ointment comprises at least one butter. The term “butter” is intended to mean a fatty substance of solid or pasty consistency at ambient temperature (25° C.) and ambient atmospheric pressure (760 mmHg).

As butters that can be used in the present invention, mention may be made of cocoa butter, shea butter and copra butter, shea butter being preferred. The amount of butters that can be used is from 0.01% to 10% by weight, preferably from 0.01% to 5% by weight. Preferably according to the invention, the butter used will be shea butter, which in particular has excellent tolerance.

It is the petroleum jelly, with the combination of a butter, in particular shea butter, and of a liquid fatty substance, in particular sweet almond oil, in the anhydrous oleaginous ointment, which allows optimized release of the active agent, in particular {4-[6-ethyl-4′-(1-ethyl-1-hydroxypropyl)-2′-propylbiphenyl-3-yloxy-methyl]-2-hydroxymethylphenyl}methanol, while at the same time providing very good tolerance of the finished product.

Waxes can also be used in the compositions according to the invention; they are used for their thickening properties and are chosen from the group consisting of waxes of animal, plant, mineral or synthetic origin, and mixtures thereof.

The term “wax” is intended to mean, in general, a lipophilic compound which is solid at ambient temperature (25° C.), having a reversible solid/liquid state change, and has a melting point which is greater than or equal to 30° C. and may range up to 200° C. and in particular up to 120° C.

According to a specific embodiment, the wax can be chosen from hydrocarbon-based compounds of the type which are glyceryl esters of saturated and unsaturated, especially polyunsaturated, fatty acids having in particular from 10 to 24 carbon atoms, unsaturated fatty acids and in particular from polyunsaturated fatty acids.

As hydrocarbon-based waxes of the type which are esters of glycerides and of polyunsaturated fatty acids, that can be used in the compositions according to the invention, mention may in particular be made of the atomized glyceryl dipalmitostearate (C₁₆-C₁₈) sold under the name “Précirol ATO 5®” by the company Gattefosse, the atomized glyceryl behenate (C₂₂) sold, for example, under the name “Compritol®888” by the company Gattefosse, and mixtures thereof.

Use may also be made of hydrocarbon-based waxes such as beeswax, lanolin wax and China insect waxes; rice wax, carnauba wax, candelilla wax, ouricury wax, alfa wax, cork fibre wax, sugarcane wax, Japan wax and sumac wax; montan wax, microcrystalline waxes, paraffins and ozokerite; polyethylene waxes, waxes obtained by Fischer-Tropsch synthesis and waxy copolymers, and also esters thereof.

Mention may also be made of waxes obtained by catalytic hydrogenation of animal or vegetable oils having C₈-C₃₂ linear or branched fatty chains. Among these waxes, mention may in particular be made of hydrogenated jojoba oil, isomerized jojoba oil such as the trans-isomerized, partially hydrogenated jojoba oil manufactured or sold by Desert Whale under the commercial reference Iso-Jojoba-50®, hydrogenated sunflower oil, hydrogenated castor oil, hydrogenated copra oil and hydrogenated lanolin oil, the di(1,1,1-trimethylolpropane) tetrastearate sold under the name Hest 2T-4S by the company Heterene, and the di(1,1,1-trimethylolpropane) tetrabehenate sold under the name Hest 2T-4B by Heterene.

Mention may also be made of silicone waxes and fluoro waxes.

Use may also be made of the wax obtained by hydrogenating esterified olive oil with stearyl alcohol that is sold under the name “Phytowax Olive 18 L 57” or else waxes obtained by hydrogenating esterified castor oil with cetyl alcohol, these waxes being sold under the name “Phytowax ricin 16L64 and 22L73” by the company Sophim. Such waxes are described in application FR-A-2792190.

According to a preferred embodiment of the invention, the thickener is beeswax, hydrogenated castor oil, carnauba wax, alkylmethylsiloxane wax (“ST wax 30”) or candelilla wax.

The amount of waxes that can be used in the composition according to the invention is from 0.01% to 10% by weight, preferably from 0.01% to 5% by weight.

The composition according to the invention can also contain the active ingredient solubilized in a solvent.

The solvent according to the present invention is chosen from pharmaceutically acceptable compounds, i.e. compounds whose use is in particular compatible with application to the skin, the mucous membranes and/or the keratin fibres. It is generally fluid, and in particular liquid, at ambient temperature.

By way of solvent agents according to the invention, mention may in particular be made of propylene glycol, PEG 400, ethanol, in particular absolute ethanol, ethoxydiglycol, sold under the name “Transcutol”, hydrogenated castor oil PEG 40, sold under the name “Cremophor RH40” by BASF, PPG-15 stearyl ether, sold under the name “Arlamol E” by Uniquema, oleyl macrogol 6 glycerides, sold under the name “Labrafil M1944CS” by the company Gattefosse, octyldodecanol, sold under the name “Eutanol G”, N-methyl-2-pyrrolidone, sold under the name “Pharmasolve”, and macrogol-15-hydroxystearate, sold under the name “Solutol HS15” by BASF, and mixtures thereof. The preferred solvent is propylene glycol.

The solvent agent is generally present in the compositions of the invention in an amount that is, firstly, sufficient to obtain the required solubility of the active ingredient to be formulated and, secondly, compatible with the need to preserve sustained chemical stability of this same active ingredient. In other words, the solvent agent must be chemically inert with respect to the active ingredient.

Advantageously, the amount of solvent used to solubilize the active ingredient, in particular {4-[6-ethyl-4′-(1-ethyl-1-hydroxypropyl)-2′-propylbiphenyl-3-yloxymethyl]-2-hydroxymethylphenyl}methanol in a composition of the invention is from 5% to 30% by weight relative to the total weight of the composition, preferably from 5% to 20% by weight.

The composition according to the invention can also comprise various other ingredients. The choice of these additional ingredients, along with that of the respective amounts thereof, is made in such a way as not to be detrimental to the expected properties for the composition. In other words, these compounds should not affect the chemical stability of the active ingredient (vitamin D or derivatives), in particular {4-[6-ethyl-4′-(1-ethyl-1-hydroxypropyl)-2′-propyl-biphenyl-3-yloxymethyl]-2-hydroxymethylphenyl}methanol, or its solubility.

The composition of the invention can comprise a lipophilic anti-irritant. By way of example, mention may be made of DL-alpha-tocopheryl acetate, oil of Melaleuca alternifolia, green tea extract and calendula extract. This agent is preferably present in an amount of between 0.001% and 2% by weight relative to the total weight of the composition, preferably between 0.001% and 1% by weight.

According to an advantageous embodiment, the composition of the invention can also comprise an antioxidant chosen from the group consisting of butylated hydroxytoluene (BHT), butylated hydroxyanisole (BHA), DL-alpha-tocopherol and propyl gallate. The amount of the antioxidant in the composition is preferably between 0.001% and 0.5% by weight, preferably between 0.002% and 0.05% by weight.

Finally, the composition according to the invention can comprise one or more pharmaceutical excipients suitable for topical application.

The present invention also relates to the use of vitamin D or of one of its derivatives of general formula (I), in particular {4-[6-ethyl-4′-(1-ethyl-1-hydroxypropyl)-2′-propylbiphenyl-3-yloxymethyl]-2-hydroxymethylphenyl}methanol for the preparation of an anhydrous pharmaceutical composition in accordance with the present description, characterized in that said composition is for use in the treatment of psoriasis and other skin disorders.

The examples hereinafter illustrate the invention but do not limit it in any way.

### EXAMPLE 1

### Compositions

In the following text, the active agent is {4-[6-ethyl-4′-(1-ethyl-1-hydroxypropyl)-2′-propylbiphenyl-3-yloxy-methyl]-2-hydroxymethylphenyl]methanol.

The percentages are given by weight relative to the total weight of the composition (m/m).

(i) Composition 1

| PHASES | INCI NAME | % |
|---|---|---|
| A | Petroleum jelly | qs 100 |
| A | Steareth 2 | 5 |
| A | Liquid paraffin | 5 |
| B | DL-alpha-tocopherol | 0.002 |
| C | Disodium edetate | 0.0065 |
| C | Disodium phosphate dihydrate | 0.026 |
| C | Purified water | 2.6 |
| D | Propylene glycol | 10 |
| D | Active agent | 0.1 |

(ii) Composition 2

| PHASES | INCI NAME | % |
|---|---|---|
| A | Petroleum jelly | qs 100 |
| A | Beeswax PEG-8 | 15 |
| A | Liquid paraffin | 5 |
| B | DL-alpha-tocopherol | 0.05 |
| B | DL-alpha-tocopheryl acetate | 1 |
| C | Disodium edetate | 0.0065 |
| C | Purified water | 2.6 |
| D | Propylene glycol | 10 |
| D | Active agent | 0.1 |

### Procedure for Compositions (i) and (ii)

The formulation makes it possible to incorporate all the constituents at a high temperature for which the petroleum jelly is liquid, and thus allow good mixing of the constituents. This also makes it possible to obtain good stability at 30° C., without any exudate.

The preparation is carried out under inactinic light.

The method is carried out in a water bath which makes it possible to maintain a homogeneous temperature over the course of the preparation.

The method is carried out using a butterfly blade, which allows effective circulation within pasty products, thereby ensuring effective homogenization.

### a) First Step: Preparation of Fatty Phase A

Phase A is weighed out into a beaker.

The beaker is heated to 75° C. in a water bath with gentle Rayneri (butterfly blade) stirring.

The stirring is maintained at 75° C. for 5 min. As soon as the starting materials have melted, the temperature is cooled to 60° C.

### b) Second Step: Preparation of Fatty Phase B

Phase B is weighed out.

### c) Third Step: Preparation of Aqueous Phase C

The starting materials are solubilized in pure water at ambient temperature with magnetic stirring. The stirring is maintained until solubilization is complete.

### d) Fourth Step: Preparation of Active Phase D

The active agent ({4-[6-ethyl-4′-(1-ethyl-1-hydroxy-propyl)-2′-propylbiphenyl-3-yloxymethyl]-2-hydroxy-methylphenyl}methanol) is solubilized in propylene glycol at ambient temperature with magnetic stirring. Homogenization is performed until complete solubilization of the active agent.

### e) Mixing

Phase B is introduced into phase A at 60° C.

Phase C is heated to 60° C. and is poured into the fatty phase (A+B) with stirring at a speed of 500 rpm.

The stirring is maintained at 60° C. for 5 min.

The temperature is cooled to 50° C., phase D is introduced and the stirring is maintained at 500 rpm for 5 min at 50° C.

The temperature is cooled to 30° C. while maintaining the stirring.

Packaging is carried out at 30° C., a temperature at which the composition has not yet completely solidified.

(iii) Composition 3 (According to the Invention)

| PHASES | INCI NAME | % |
|---|---|---|
| A | Petroleum jelly | qs 100 |
| A | Beeswax | 5 |
| B | Sweet almond oil (Prunus | 10 |
| | amygdalus dulcis) | |
| B | DL-alpha-tocopherol | 0.05 |
| B | DL-alpha-tocopheryl acetate | 1 |
| B | Shea butter | 5 |
| D | Propylene glycol | 10 |
| D | Active agent | 0.1 |

Procedure for Composition (iii)

The preparation is carried out under inactinic light.

### a) First Step: Preparation of Fatty Phase A

Phase A is weighed out into a beaker.

The beaker is heated to 75° C. in a water bath with gentle Rayneri (butterfly blade) stirring.

The stirring is maintained at 75° C. for 5 min. As soon as the starting materials have melted, the temperature is cooled to 60° C.

### b) Second Step: Preparation of Fatty Phase B

Phase B is weighed out. Phase B is heated to 60° C. and homogenized with magnetic stirring.

### c) Third Step: Preparation of Active Phase D

The active agent ({4-[6-ethyl-4′-(1-ethyl-1-hydroxypropyl)-2′-propylbiphenyl-3-yloxymethyl]-2-hydroxymethylphenyl}methanol) is solubilized in propylene glycol at ambient temperature with magnetic stirring. Homogenization is carried out until complete solubilization of the active agent.

### d) Mixing

Phase B is introduced into phase A at 60° C. with Rayneri stirring at a speed of 300 rpm.

The temperature is cooled to 50° C. and phase D is poured into the fatty phase (A+B) with Rayneri stirring at 500 rpm. The mixture is left at 50° C. for 5 min with stirring.

The temperature is cooled to 30° C.

Packaging is carried out at 30° C., a temperature at which the composition has not yet completely solidified.

### EXAMPLE 2

### Study of Tolerance of the Compositions of the Invention

Throughout the following text, the expression “vehicle for formulating a composition” is intended to mean the composition without active ingredient.

(i) A tolerance study was carried out on the vehicles for formulating compositions 2 and 3 compared with the vehicle for composition 1, known for its high tolerance.

Treatment: one application daily from day 1 to day 6 of 20 μl of composition is made to the right ear of Balb/c mice.

Evaluation method: clinical observation and measurement of the thickness of the mouse ear from day 2 to day 12. Weighing of the animals on day 1 and on day 12.

Conclusion:

The vehicles for compositions 1 and 3 are not irritant, the vehicle for composition 2 appears to be irritant (increase in thickness of the ear).

(ii) A study of tolerance was also carried out on compositions 1 to 3 which contain 0.1% (m/m) of active agent, in parallel with a composition containing 0.1% of active agent in ethanol.

The same treatment and the same evaluation method as above are applied.

Conclusion:

Compositions 1 and 3 induce the same response profile with an amplitude that is approximately 30% less than that of the active agent at 0.1% in ethanol.

None of the vehicles induces an inflammatory response, none of the compositions tested induces any blood-calcium-raising effect or any weight loss.

From the above, it appears that the anhydrous three-way combination of petroleum jelly with a liquid fatty substance and a butter (vehicle for composition 3) according to the invention confers high tolerance on the formula.

### EXAMPLE 3

### Study of Release/Penetration

Objective: to compare the in vitro percutaneous absorption of the radiolabelled active agent through human skin, at 0.1% (m/m) in various formulations. Compositions 1 and 3 give the best results in terms of release/penetration of the active agent.

Composition 2 gives the worst result.

Anhydrous composition 3, comprising the three-way combination of petroleum jelly with a liquid fatty substance and a butter, therefore has good properties of release/penetration of the active agent in the skin.

### EXAMPLE 4

### Solubility of the Active Agent

### Maximum Solubility of the Active Agent in Various Excipients

| | Sol max |
|---|---|
| Excipients | (% w/w) |
| Propylene glycol | 2.3351 |
| Ethanol 95 | >20 |
| PEG 400 | 6.894 |
| Transcutol | >20 |
| Sweet almond oil | 0.0932 |
| Cremophor RH40 | 3.989 |
| Arlamol E | 1.033 |
| Labrafil | 0.936 |
| M1944CS | |
| Eutanol G | 0.322 |
| Miglyol 812 | 0.3167 |
| IPP | 0.1654 |
| Mirasil CM5 | NA |
| Primol 352 | 0.0009 |

### EXAMPLE 5

### Stability of Compositions 1 to 3

The physical stability of compositions 1 to 3 is evaluated by macroscopic and microscopic observation of the composition at ambient temperature, at 4° C. and at 30° C. after 1 month, 2 months and 3 months.

At ambient temperature, the macroscopic observation makes it possible to guarantee the physical integrity of the products and the microscopic observation makes it possible to verify that there is no recrystallization of the solubilized active agent.

The characterization of each of the final compositions is completed by measuring the flow threshold. A Haake VT550 rheometer is used with an SVDIN measuring spindle. The rheograms are carried out at 25° C. and at a shear rate of 4 s⁻¹ (γ), and by measuring the shear stress. The term “flow threshold” (τ0 expressed in Pascals) is intended to mean the force required (minimum shear stress) to overcome the cohesion forces of Van der Waals type and bring about flow. The flow threshold is related to the value found at the shear rate of 4 s⁻¹.

These measurements are carried out at T0, after 1 month, 2 months and 3 months.

### Composition 1: SPECIFICATIONS T0:

Macroscopic appearance: thick translucent shiny ointment.

Microscopic appearance: refringent network (yellow, violet, blue) characteristic of the petroleum jelly network.

Centrifugation: 30 min at 3000 rpm NTR* NTR: nothing to report
15 min at 10 000 rpm Release

Viscosity: Tau 0: 346 Pa.s⁻¹

Analytical assay: T0 yield=100.2%

| | | T1 month | T2 months | T3 months |
|---|---|---|---|---|
| AT | Macroscopic | Complies | Complies | Complies |
| | appearance | | | |
| | Centrifugation | Complies | Complies | Complies |
| | Viscosity Tau | No | No | 291 |
| | 0 (Pa · s−¹) | measurement | measurement | |
| | Analytical | 101% | 99.4% | 99.4% |
| | assay | | | |
| 4° C. | Macroscopic | Complies | Complies | Complies |
| | appearance | | | |
| 30° | Macroscopic | Complies | Complies | Complies |
| | appearance | | | |
| | Analytical | 98.6% | 98.9% | 98.8% |
| | assay | | | |

### Composition 2: SPECIFICATIONS T0:

Macroscopic appearance: thick white ointment.

Microscopic appearance: refringent network (yellow, violet, blue) characteristic of the petroleum jelly network.

Centrifugation: 30 min at 3000 rpm NTR
15 min at 10 000 rpm NTR

Viscosity: Tau 0: 434 Pa.s⁻¹

Analytical assay: T0 yield=99.1%

| | | T1 month | T2 months | T3 months |
|---|---|---|---|---|
| AT | Macroscopic | Complies | Complies | Complies |
| | appearance | | | |
| | Centrifugation | Complies | Complies | Complies |
| | Viscosity Tau | No | No | 413 |
| | 0 (Pa · s⁻¹) | measurement | measurement | |
| | Analytical | 99.4% | 100.2% | 98.5% |
| | assay | | | |
| 4° C. | Macroscopic | Complies | Complies | Complies |
| | appearance | | | |
| 30° C. | Macroscopic | Complies | Presence of | Complies |
| | appearance | | seepage | |
| | Viscosity Tau | No | No | 456 |
| | 0 (Pa · s⁻¹) | measurement | measurement | |
| | Analytical | 101.2% | 98% | 101.1% |
| | assay | | | |

### Composition 3: SPECIFICATIONS T0:

Macroscopic appearance: thick, shiny, pale yellow ointment.

Microscopic appearance: refringent network (yellow, violet, blue) characteristic of the petroleum jelly network.

Centrifugation: 30 min at 3000 rpm NTR
15 min at 10 000 rpm seepage

Viscosity: Tau 0: 369 Pa.s⁻¹

Analytical assay: T0 yield=97.1%

| | | T1 month | T2 months | T3 months |
|---|---|---|---|---|
| AT | Macroscopic | Complies | Complies | Complies |
| | appearance | | | |
| | Centrifugation | Complies | Complies | Complies |
| | Viscosity Tau | No | No | 228 |
| | 0 (Pa · s⁻¹) | measurement | measurement | |
| | Analytical | 101.2% | 99.9% | 99.3% |
| | assay | | | |
| 4° C. | Macroscopic | Complies | Complies | Complies |
| | appearance | | | |
| 30° C. | Macroscopic | Complies | Presence of | Complies |
| | appearance | | seepage | |
| | Viscosity Tau | No | No | 340 |
| | 0 (Pa · s⁻¹) | measurement | measurement | |
| | Analytical | 99.2% | 97% | 101.1% |
| | assay | | | |

1. Anhydrous pharmaceutical composition, characterized in that it comprises:
a) an oleaginous ointment comprising petroleum jelly and a combination of emollients comprising at least one liquid fatty substance and at least one butter, and
b) as active ingredient, a compound chosen from vitamin D and its derivatives, of general formula (I) below: 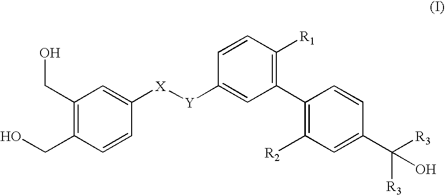
in which:
X—Y represents a bond chosen from the following structures:
—CH₂—CH₂—
—CH₂—O—
—O—CH₂—
—CH₂—N(R₄)—
R₄ having the meanings given hereinafter,
R₁ represents a methyl radical or an ethyl radical,
R₂ represents an ethyl radical, a propyl radical or an isopropyl radical,
R₃ represents an ethyl radical or a trifluoromethyl radical,
R₄ represents a hydrogen atom, a methyl radical, an ethyl radical or a propyl radical,
said active agent being in a solubilized form in said composition.

2. Composition according to claim 1, characterized in that the active ingredient is chosen from the following compounds:
1—{5-[4′-(1-ethyl-1-hydroxypropyl)-6-methyl-2′-propylbiphenyl-3-yloxymethyl]-2-hydroxy-methylphenyl}methanol;
2—{5-[6,2′-diethyl-4′-(1-ethyl-1-hydroxy-propyl)biphenyl-3-yloxymethyl]-2-hydroxy-methylphenyl}methanol;
3—{4-[6-ethyl-4′-(1-ethyl-1-hydroxypropyl)-2′-propylbiphenyl-3-yloxymethyl]-2-hydroxy-methylphenyl}methanol;
4—{4-[6-ethyl-4′-(1-ethyl-1-hydroxypropyl)-2′-isopropylbiphenyl-3-yloxymethyl]-2-hydroxy-methylphenyl}methanol;
5—(4-(2-[4′-(1-ethyl-1-hydroxypropyl)-6-methyl-2′-propylbiphenyl-3-yl]ethyl}-2-hydroxy-methylphenyl)methanol;
6—{4-[4′-(1-ethyl-1-hydroxypropyl)-6-methyl-2′-propylbiphenyl-3-ylmethoxy]-2-hydroxymethyl-phenyl}methanol;
7—(4-{[4′-(1-ethyl-1-hydroxypropyl)-6-methyl-2′-propylbiphenyl-3-ylamino]methyl)-2-hydroxymethylphenyl)methanol;
8—[4-({[4′-(1-ethyl-1-hydroxypropyl)-6-methyl-2′-propylbiphenyl-3-yl]methylamino}methyl)-2-hydroxymethylphenyl]methanol;
9—[4-({ethyl-[4′-(1-ethyl-1-hydroxypropyl)-6-methyl-2′-propylbiphenyl-3-yl]amino}methyl)-2-hydroxymethylphenyl]methanol;
10—[4-({[4′-(1-ethyl-1-hydroxypropyl)-6-methyl-2′-propylbiphenyl-3-yl]propylamino}methyl)-2-hydroxymethylphenyl]methanol;
11—(2-hydroxymethyl-4-{2-[6-methyl-2′-propyl-4′-(2,2,2-trifluoro-1-hydroxy-1-trifluoromethyl-ethyl)biphenyl-3-yl]ethyl}phenyl)methanol;
12—{2-hydroxymethyl-4-[6-methyl-2′-propyl-4′-(2,2,2-trifluoro-1-hydroxy-1-trifluoromethyl-ethyl)biphenyl-3-yloxymethyl]phenyl}methanol;
13—{2-hydroxymethyl-4-[6-methyl-2′-propyl-4′-(2,2,2-trifluoro-1-hydroxy-1-trifluoromethyl-ethyl)biphenyl-3-ylmethoxy]phenyl}methanol;
14—(2-hydroxymethyl-4-{[6-methyl-2′-propyl-4′-(2,2,2-trifluoro-1-hydroxy-1-trifluoromethyl-ethyl)biphenyl-3-ylamino]methyl}phenyl)-methanol;
15—[2-hydroxymethyl-4-({N-methyl[6-methyl-2′-propyl-4′-(2,2,2-trifluoro-1-hydroxy-1-trifluoromethylethyl)biphenyl-3-yl]amino}-methyl)phenyl]methanol;
16—[4-({N-ethyl[6-methyl-2′-propyl-4′-(2,2,2-trifluoro-1-hydroxy-1-trifluoromethylethyl)-biphenyl-3-yl]amino}methyl)-2-hydroxymethyl-phenyl]methanol;
17—[2-hydroxymethyl-4-({[6-methyl-2′-propyl-4′-(2,2,2-trifluoro-1-hydroxy-1-trifluoromethyl-ethyl)biphenyl-3-yl]N-propylamino}methyl)-phenyl]methanol;
18—(4-{2-[6-ethyl-4′-(1-ethyl-1-hydroxypropyl)-2′-propylbiphenyl-3-yl]ethyl}-2-hydroxy-methylphenyl)methanol;
19—{4-[6-ethyl-4′-(1-ethyl-1-hydroxypropyl)-2′-propylbiphenyl-3-ylmethoxy]-2-hydroxymethyl-phenyl}methanol;
20—(4-{[6-ethyl-4′-(1-ethyl-1-hydroxypropyl)-2′-propylbiphenyl-3-ylamino]methyl}-2-hydroxy-methylphenyl)methanol;
21—[4-({[6-ethyl-4′-(1-ethyl-1-hydroxypropyl)-2′-propylbiphenyl-3-yl]methylamino}methyl)-2-hydroxymethylphenyl]methanol;
22—[4-({ethyl-[6-ethyl-4′-(1-ethyl-1-hydroxy-propyl)-2′-propylbiphenyl-3-yl]amino}methyl)-2-hydroxymethylphenyl]methanol;
23—[4-({[6-ethyl-4′-(1-ethyl-1-hydroxypropyl)-2′-propylbiphenyl-3-yl]propylamino}methyl)-2-hydroxymethylphenyl]methanol;
24—(4-{2-[6-ethyl-2′-propyl-4′-(2,2,2-trifluoro-1-hydroxy-1-trifluoromethylethyl)biphenyl-3-yl]ethyl}-2-hydroxymethylphenyl)methanol;
25—{4-[6-ethyl-2′-propyl-4′-(2,2,2-trifluoro-1-hydroxy-1-trifluoromethylethyl)biphenyl-3-yloxymethyl]-2-hydroxymethylphenyl}methanol;
26—{4-[6-ethyl-2′-propyl-4′-(2,2,2-trifluoro-1-hydroxy-1-trifluoromethylethyl)biphenyl-3-ylmethoxy]-2-hydroxymethylphenyl}methanol;
27—(4-{[6-ethyl-2′-propyl-4′-(2,2,2-trifluoro-1-hydroxy-1-trifluoromethylethyl)biphenyl-3-ylamino]methyl}-2-hydroxymethylphenyl)-methanol;
28—[4-({[6-ethyl-2′-propyl-4′-(2,2,2-trifluoro-1-hydroxy-1-trifluoromethylethyl)biphenyl-3-yl]methylamino}methyl)-2-hydroxymethyl-phenyl]methanol;
29—[4-({N-ethyl[6-ethyl-2′-propyl-4′-(2,2,2-trifluoro-1-hydroxy-1-trifluoromethylethyl)-biphenyl-3-yl]amino}methyl)-2-hydroxymethyl-phenyl]methanol;
30—[4-({[6-ethyl-2′-propyl-4′-(2,2,2-trifluoro-1-hydroxy-1-trifluoromethylethyl)biphenyl-3-yl]-N-propylamino}methyl)-2-hydroxymethyl-phenyl]methanol;
31—(4-{[4′-(1-ethyl-1-hydroxypropyl)-6,2′-dimethylbiphenyl-3-ylamino]methyl}-2-hydroxymethylphenyl)methanol.

3. Composition according to claim 2, characterized in that the active ingredient is {4-[6-ethyl-4′-(1-ethyl-1-hydroxypropyl)-2′-propylbiphenyl-3-yloxymethyl]-2-hydroxymethylphenyl}methanol.

4. Composition according to any one of claims 1 to 3, characterized in that the liquid fatty substance is chosen from liquid paraffins, sweet almond oil, palm oil, soya oil, sesame oil, sunflower oil, lanolin, squalene, fish oil, mink oil, cetearyl isononanoate, diisopropyl adipate, isopropyl palmitate and caprylic/capric triglyceride.

5. Composition according to one of claims 1 to 4, characterized in that the butter is chosen from shea butter, copra butter and cocoa butter.

6. Composition according to one of claims 1 to 5, characterized in that the ointment comprises petroleum jelly, a liquid fatty substance and a butter.

7. Composition according to claim 6, characterized in that the liquid fatty substance is sweet almond oil and the butter is shea butter.

8. Composition according to one of claims 1 to 7, characterized in that it is for topical application.

9. Composition according to one of claims 1 to 8, characterized in that it has a water content of less than or equal to 5% by weight relative to the total weight of the composition, in particular less than or equal to 3%, and especially equal to zero.

10. Composition according to any one of claims 1 to 9, characterized in that the active ingredient is solubilized in a solvent.

11. Composition according to claim 10, characterized in that the solvent is chosen from the group consisting of propylene glycol, PEG-400, ethanol, ethoxydiglycol, polyoxyl 40 hydrogenated castor oil, PPG-15 stearyl ether, oleyl macrogol 6 glycerides, octyldodecanol, N-methyl-2-pyrrolidone, macrogol-15 hydroxystearate, and mixtures thereof.

12. Composition according to any one of claims 1 to 11, characterized in that the amount of active ingredient in a solubilized form is from 0.0001% to 5% by weight relative to the total weight of the composition, preferably from 0.001% to 1% by weight, and more particularly from 0.05% to 0.2% by weight.

13. Use of vitamin D or of one of its derivatives of general formula (I), for the preparation of an anhydrous pharmaceutical composition according to any one of claims 1 to 12, said composition being for use in the treatment of psoriasis and other skin disorders.
